(19) United States
(12) Patent Application Publication
(10) Pub. No.: US 2007/0259346 A1
(43) Pub. Date: Nov. 8, 2007

(54) Analysis of arrays

(75) Inventors: David B. Gordon, Somerville, MA (US); Andrew Payne, Lincoln, MA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Loveland, CO (US)

(21) Appl. No.: 11/417,348

(22) Filed: May 3, 2006

(51) Int. Cl. C12Q 1/68; G06F 19/00

(52) U.S. Cl. 435/6; 702/20

(57) ABSTRACT

Techniques and equipment are provided, including computer-related software and hardware, for analyzing biological events on multiple-spot arrays for chemical, biological, or biochemical analysis. In one arrangement, techniques and equipment are provided for analyzing nucleotide sequences of nucleic acid molecules, e.g., using multiple probes per spot of an array. Arrays can be provided in which multiple probes exist on multiple spots, and where analysis of whether a particular target molecule bound to a particular spot does not necessarily provide information on what probe the target bound to. The invention provides techniques for analyzing biological events in the form of, for example, deconvolution of binding data.

### BACKGROUND

Arrays of nucleic acids have become an increasingly important tool in the biotechnology industry and related fields. These nucleic acid arrays, in which a plurality of distinct or different nucleic acids are positioned on a solid support surface in the form of an array or pattern, find use in a variety of applications, including gene expression analysis, nucleic acid synthesis, drug screening, nucleic acid sequencing, mutation analysis, array CGH, location analysis (also known as ChIP-Chip), and the like.

Arrays having a large number of spots are advantageous in that large genomes or transcriptomes can be assayed at higher resolutions and/or with fewer number of slides per experiment. Current methods of increasing the density of spots per array include forming spots with smaller surface areas and/or positioning spots closer together on the array. Although these methods may be useful, other methods of increasing the effective probe density of arrays would be beneficial.

Arrays having a large number of spots can provide significant information per unit area of array and, as that information density increases, the arrays can become more complex and can require more sophisticated analysis to translate basic information readable from an array to fundamental information which the array is designed to provide.

While high-density arrays are known for use in many chemical, biological, and biochemical arenas, more sophisticated arrays, and techniques for their use, would be desirable.

### SUMMARY OF THE INVENTION

Systems and methods for processing to decode information from a microarray are provided. In one embodiment, a method is provided. In certain embodiments, the method comprises a) reading an array comprising a plurality of spots that each contain nucleic acid sequences that hybridize to non-contiguous genomic regions to identify spots that produce a signal, b) reading a design file to identify information on the sequences and/or the chromosomal binding sites of nucleic acid sequences, and c) decoding said information to identify a biological phenomenon.

In some embodiments, the plurality of features comprise a mixture of oligonucleotides having different sequences. In one embodiment, the plurality of features comprise an oligonucleotide comprising a plurality of hybridizing segments. The oligonucleotide can include, for example, at least a first and a second hybridizing segment that are contiguous on the oligonucleotide. In some cases, the oligonucleotide includes at least a first and a second hybridizing segment that are not contiguous on the oligonucleotide. In some embodiments, the oligonucleotide is, at least 60, at least 80, at least 100, at least 150 or at least 200 nucleotides in length. In another embodiment, the hybridizing segments are each at least 10 bases, at least 20 bases, at least 30 bases, at least 40 bases, or at least 50 bases in length. The oligonucleotide may comprise, for example, at least 2, at least 3, at least 4, or at least 5 hybridizing segments.

In another embodiment, a method is provided. The method comprises acts of reading spot signals associated with spots of an array or array set, wherein each spot, for at least some of the spots of the array or array set, include at least first and second oligonucleotide probes having respective nucleotide sequences. The method also comprises reading a design file comprising parameters including the nucleotide sequence of each probe within said spots of the array or array set, and/or the location of each probe in terms of chromosomal coordinates if the probes were hybridized to a nucleic acid molecule of interest, associating a corresponding spot signal with a parameter from the design file, and for at least some of the spots including those corresponding to first and second oligonucleotide probes per spot, processing to decode information identifying a biological phenomenon in the nucleic acid molecule of interest. In some cases, the biological phenomenon can be the presence or absence of protein binding on the nucleic acid molecule of interest.

The method may further comprise, from one or more readings of spot signals, creating a file including values of spot signals associated with the spots. In some instances, at least a plurality of spots of the array include compound oligonucleotide probes. The compound probes have an average length of at least 100 nucleotides. In some embodiments, at least a plurality of the compound probes comprise at least first and second oligonucleotide probes contiguous on the compound probe. In other embodiments, a plurality of the spots comprise multiple, non-contiguous probes.

In one particular embodiment, the biological phenomenon is identified if and only if all of the spots comprising probes relating to that phenomenon show a signal. In another embodiment, the biological phenomenon is identified if and only if all of the spots comprising probes in the genomic neighborhood of the phenomenon show a signal.

In another embodiment, an article is provided. The article comprises a machine-readable medium having a program stored thereon, which program has instructions for, when executed, performing acts of analyzing values of spot signals associated with spots of an array or array set, wherein each spot signal, for at least some of the spot signals, is associated with at least first and second oligonucleotide probes, and wherein a value of a first spot signal corresponds to the first oligonucleotide probe only if the joint significance of the value of the first spot and a value of at least a second spot signal from at least a second spot, which comprises the first, but not the second, oligonucleotide probe, is significant.

In another embodiment, another article is provided. The article comprises a machine-readable medium having a program stored thereon, which program has instructions for, when executed, performing acts of analyzing values of spot signals associated with spots of an array or array set, wherein each spot signal, for at least some of the spot signals, is associated with at least first and second oligonucleotide probes, and wherein a value of a first spot signal corresponds to the first probe only if values of spot signals from spots comprising probes that are genomic neighbors of the first probe, together with the value of the first spot signal, produce an expected distribution of values.

In yet another embodiment, a system is provided. The system comprises a scanner for reading spot signals from an array or array set including a plurality of spots, wherein each spot, for at least some of the spots of the array or array set, include at least first and second oligonucleotide probes having respective nucleotide sequences, and a processor for receiving output from the scanner and executing operations to analyze the scanner output and providing an indication of a biological phenomenon in a nucleic acid molecule of interest.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram of a microarray including a plurality of spots comprising compound probes according to another embodiment of the invention;

FIG. 2 shows deconvolution of signals produced from the microarray of FIG. 1 according to another embodiment of the invention;

FIG. 3 is a schematic diagram of another microarray including a plurality of spots comprising compound probes according to another embodiment of the invention;

FIGS. 4A-4E are deconvolution of signals produced after hybridization in the microarray of FIG. 3 according to another embodiment of the invention;

FIGS. 5A and 5B show data illustrating fitting of intensities to the shape of an expected distribution according to another embodiment of the invention;

FIGS. 6A and 6B show data illustrating post-processing of signals using a filter constructed to reduce high signals that are inconsistent with neighboring signals, according to another embodiment of the invention;

FIG. 7 is a schematic diagram showing the interrelationship between a probe scanner, a processor, a design file, and a display according to another embodiment of the invention; and

FIG. 8 is a schematic diagram showing acts associated with the process of decoding signals from an array according to another embodiment of the invention.

### DEFINITIONS

Unless defined otherwise, all technical and scientific terms used herein have the same meaning as commonly understood by one of ordinary skill in the art to which this invention belongs. Certain elements are defined below for the sake of clarity and ease of reference.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “mRNA” means messenger RNA.

The term “biomolecule” means any organic or biochemical molecule, group or species of interest that may be formed in an array on a substrate surface. Exemplary biomolecules include peptides, proteins, amino acids and nucleic acids.

The term “peptide” as used herein refers to any compound produced by amide formation between a carboxyl group of one amino acid and an amino group of another group.

The term “oligopeptide” as used herein refers to peptides with fewer than about 10 to 20 residues, i.e., amino acid monomeric units.

The term “polypeptide” as used herein refers to peptides with more than 10 to 20 residues.

The term “protein” as used herein refers to polypeptides of specific sequence of more than about 50 residues.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “polynucleotide” or “nucleic acid” refers to a polymer composed of nucleotides, natural compounds such as deoxyribonucleotides or ribonucleotides, or compounds produced synthetically (e.g., PNA as described in U.S. Pat. No. 5,948,902 and the references cited therein), which can hybridize with naturally-occurring nucleic acids in a sequence specific manner analogous to that of two naturally occurring nucleic acids, e.g., can participate in Watson-Crick base pairing interactions. The polynucleotide can have from about 20 to 5,000,000 or more nucleotides. The larger polynucleotides are generally found in the natural state. In an isolated state the polynucleotide can have about 30 to 50,000 or more nucleotides, usually about 100 to 20,000 nucleotides, more frequently 500 to 10,000 nucleotides. Isolation of a polynucleotide from the natural state often results in fragmentation. It may be useful to fragment longer target nucleic acid sequences, particularly RNA, prior to hybridization to reduce competing intramolecular structures.

The polynucleotides include nucleic acids, and fragments thereof, from any source in purified or unpurified form including DNA (dsDNA and ssDNA) and RNA, including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. The polynucleotide can be only a minor fraction of a complex mixture such as a biological sample. Also included are genes, such as hemoglobin gene for sickle-cell anemia, cystic fibrosis gene, oncogenes, cDNA, and the like.

The polynucleotide can be obtained from various biological materials by procedures well known in the art. The polynucleotide, where appropriate, may be cleaved to obtain a fragment that contains a target nucleotide sequence, for example, by shearing or by treatment with a restriction endonuclease or other site-specific chemical cleavage method.

For purposes of this invention, the polynucleotide, or a cleaved fragment obtained from the polynucleotide, will usually be at least partially denatured or single stranded or treated to render it denatured or single stranded. Such treatments are well known in the art and include, for instance, heat or alkali treatment, or enzymatic digestion of one strand. For example, dsDNA can be heated at 90 to 100 degrees Celcius for a period of about 1 to 10 minutes to produce denatured material.

The nucleic acids may be generated by in vitro replication and/or amplification methods such as the Polymerase Chain Reaction (PCR), asymmetric PCR, the Ligase Chain Reaction (LCR) and so forth. The nucleic acids may be either single-stranded or double-stranded. Single-stranded nucleic acids are preferred because they lack complementary strands that compete for the oligonucleotide precursors during the hybridization step of the method of the invention.

The term “oligonucleotide” refers to a polynucleotide, usually single stranded, usually a synthetic polynucleotide but may be a naturally occurring polynucleotide. The length of an oligonucleotide is generally governed by the particular role thereof, such as, for example, probes (e.g., compound probes), primers, X-mers, and the like. Various techniques can be employed for preparing an oligonucleotide. Such oligonucleotides can be obtained by biological synthesis or by chemical synthesis. For short oligonucleotides (i.e., up to about 100 nucleotides), chemical synthesis will frequently be more economical as compared to the biological synthesis. In addition to economy, chemical synthesis provides a convenient way of incorporating low molecular weight compounds and/or modified bases during specific synthesis steps. Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. Methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al. (Proc. Nat. Acad. Sci. USA 91:5022-5026, 1994). In some cases, synthesis of certain oligonucleotides (e.g., compound probes) can be performed according to methods disclosed in U.S. Patent Publication No. 2005/0214779, filed Mar. 29, 2004, entitled “Methods for in situ generation of nucleic acid arrays”, which is incorporated herein by reference.

Generally, as used herein, the terms “oligonucleotide” and “polynucleotide” are used interchangeably. Further, generally, the term “nucleic acid molecule” also encompasses oligonucleotides and polynucleotides.

The term “oligonucleotide” refers to a nucleic acid that has a defined length, which is usually a sequence of at least 3 nucleotides, in some cases, 4 to 14 nucleotides, in other cases 5 to 20, 5 to 30, 8 to 50, 8 to 60, 50 to 100, 50 to 120, 50 to 150, 100-200 nucleotides in length, or longer. An oligonucleotide of a certain length X may be referred to as an X-mer. For instance, a 60-mer refers to an oligonucleotide having a sequence of 60 nucleotides.

The term “X-mer precursors”, sometimes referred to as “oligonucleotide precursors” refers to a nucleic acid sequence that is complementary to a portion of the target nucleic acid sequence. The oligonucleotide precursors are sequences of nucleoside monomers joined by phosphorus linkages (e.g., phosphodiester, alkyl and aryl-phosphate, phosphorothioate, phosphotriester), or non-phosphorus linkages (e.g., peptide, sulfamate and others). They may be natural or synthetic molecules of single-stranded DNA and single-stranded RNA with circular, branched or linear shapes, and optionally including domains capable of forming stable secondary structures (e.g., stem-and-loop and loop-stem-loop structures). The oligonucleotide precursors contain a 3′-end and a 5′-end.

The term “oligonucleotide probe” or “probe” refers to an oligonucleotide employed to hybridize to a portion of a polynucleotide such as another oligonucleotide or a target nucleotide sequence. The design and preparation of the oligonucleotide probes are generally dependent upon the sequence to which they hybridize.

The phrase “nucleic acid molecule bound to a surface of a solid support” or “probe bound to a solid support” or a “target bound to a solid support” or “polynucleotide bound to a solid support” refers to a nucleic acid molecule (e.g., an oligonucleotide or polynucleotide) or mimetic thereof (e.g., comprising at least one PNA or LNA monomer) that is immobilized on a surface of a solid substrate, where the substrate can have a variety of configurations, e.g., including, but not limited to, planar, non-planar, a sheet, bead, particle, slide, wafer, web, fiber, tube, capillary, microfluidic channel or reservoir, or other structure. In certain embodiments, collections of nucleic acid molecules are present on a surface of the same support, e.g., in the form of an array, which can include at least about two nucleic acid molecules, which may be identical or comprise a different nucleotide base composition. As used herein, the terms “bound to a solid support” and “attached to a solid support” may be used interchangeably unless context dictates otherwise.

“Addressable sets of probes” and analogous terms refer to the multiple known regions of different moieties of known characteristics (e.g., base sequence composition) supported by or intended to be supported by a solid support, i.e., such that each location is associated with a moiety of a known characteristic and such that properties of a target moiety can be determined based on the location on the solid support surface to which the target moiety hybridizes under stringent conditions.

A solid support, in some embodiments, is non-porous. In certain embodiments, a non-porous support comprises a bead. As used herein, a “non-porous support” refers to a support having a pore size that essentially excludes synthesis reagents (e.g., such as biopolymer precursors or solutions for preparing biopolymers, including but not limited to deblocking and purging solutions) from entering the support (e.g., penetrating the surface). In one aspect, to the extent there are any openings/pores in a surface of a support, the openings/pores can be less than about 100 Angstroms, less than about 60 angstroms, less than about 50 Angstroms, less than about 25 Angstroms, etc. Included in this definition are supports having these specified size restrictions or properties in their natural state or which have been treated to reduce the size of any openings/pores to obtain these restrictions/properties. In certain embodiments, supports include non-porous beads. Such beads can be fabricated as is known in the art, for example, as described in U.S. Patent Publication No. 2003/0225261.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding (or hybridization) agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

An “array set” includes one or more arrays tailored to a particular assay. An array set may include more than one array, e.g., when there are too many spots or features to fit on a single substrate and/or spots are spread over multiple substrates. The multiple substrates may be said to be part of an array set. An example of an array set includes a “10-set” product, which is on ten glass slides with about 440,000 spots (e.g., about 44 k spots per slide). An “array” and “array set” may be used interchangeably herein in some embodiments of the invention.

Any given substrate may carry any number of oligonucleotides on a surface thereof. In one embodiment, one, two, four, or more arrays are disposed on a front surface of the substrate. Depending upon the use, any, or all, of the arrays may be the same or different from one another and each may include multiple spots or features of different moieties (for example, different polynucleotide sequences). A spot or feature of an array is generally homogeneous in composition and in concentration. A region at a particular predetermined location (an “address”) on the array will detect a particular target or set of targets (although a spot or feature may incidentally detect non-targets of that spot or feature). The target for which the spot or feature is specific is, in representative embodiments, known.

A typical array may contain more than ten, more than one hundred, more than one thousand more ten thousand spots, more than one hundred thousand spots, or even more than one million spots in an area of less than 20 cm² or even less than 10 cm². For example, spots may have widths (that is, diameter, for a round spot) in the range from 10 μm to 1.0 cm. In other embodiments, each spot may have a width in the range of 1.0 μm to 1.0 mm, usually 5.0 μm to 500 μm, and more usually 10 μm to 200 μm. Non-round spots may have area ranges equivalent to that of circular spots with the foregoing width (diameter) ranges. At least some, or all, of the spots are of different compositions (for example, when any repeats of each spot composition are excluded, the remaining spots may account for at least 5%, 10%, or 20% of the total number of spots).

In some embodiments, interspot areas will typically (but not essentially) be present which do not carry any oligonucleotide (or other biopolymer or chemical moiety of a type of which the features are composed). Such interspot areas typically will be present where the arrays are formed by processes involving drop deposition of reagents but may not be present when, for example, light directed synthesis fabrication processes are used. It will be appreciated though, that the interfeature areas, when present, could be of various sizes and configurations. In other embodiments, however, oligonucleotides may be present in interspot areas. In one particular embodiment, spots are arranged adjacent one another such that there are no interspot areas between each spot.

Each array may cover an area of less than 100 cm², or even less than 50 cm², 10 cm² or 1 cm². In certain embodiments, the substrate carrying the one or more arrays will be shaped as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either oligonucleotide precursor units (such as monomers) in the case of in situ fabrication, or the previously obtained oligonucleotide. Such methods are described in detail in, for example, the previously cited references including U.S. Pat. Nos. 6,242,266; 6,232,072; 6,180,351; 6,171,797; 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

The term “biological sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained, e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g. folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The term “target nucleic acid sequence” refers to a sequence of nucleotides to be identified, detected or otherwise analyzed, usually existing within a portion or all of a polynucleotide. In the present invention, the identity of the target nucleotide sequence may or may not be known. The identity of the target nucleotide sequence may be known to an extent sufficient to allow preparation of various sequences hybridizable with the target nucleotide sequence and of oligonucleotides, such as probes and primers, and other molecules necessary for conducting methods in accordance with the present invention and so forth. Determining the sequence of the target nucleic acid includes in its definition, determining the sequence of the target nucleic acid or sequences within regions of the target nucleic acid to determine the sequence de novo, to resequence, and/or to detect mutations and/or polymorphisms. In some cases, target nucleic acid sequences are present in a biological sample of interest. The terms “target nucleic acid” and “nucleic acid molecule of interest” are used interchangeably. A target nucleic acid or a nucleic acid molecule of interest may represent, for example, a genome (e.g., a “target genome”) or a transcriptome (e.g., a “target transcriptome”).

The target sequence may contain from about 30 to 5,000 or more nucleotides, or from 50 to 1,000 nucleotides. In some cases, the target nucleotide sequence is generally a fraction of a larger molecule. In other cases, the target nucleotide sequence may be substantially the entire molecule, such as a polynucleotide as described above. The minimum number of nucleotides in the target nucleotide sequence is selected to assure that the presence of a target polynucleotide in a sample is a specific indicator for the presence of polynucleotide in a sample. The maximum number of nucleotides in the target nucleotide sequence is normally governed by several factors: the length of the polynucleotide from which it is derived, the tendency of such polynucleotide to be broken by shearing or other processes during isolation, the efficiency of any procedures required to prepare the sample for analysis (e.g., transcription of a DNA template into RNA) and the efficiency of identification, detection, amplification, and/or other analysis of the target nucleotide sequence, where appropriate.

The terms “hybridization”, and “hybridizing”, in the context of nucleotide sequences are used interchangeably herein. The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the hybridization of the two sequences. Increased stringency can be achieved by elevating the temperature, increasing the ratio of co-solvents, lowering the salt concentration, and the like. Hybridization also includes in its definition the transient hybridization of two complementary sequences. It is understood by those skilled in the art that non-covalent hybridization between two molecules, including nucleic acids, obeys the laws of mass action. Therefore, for purposes of the present invention, hybridization between two nucleotide sequences for a length of time that permits primer extension and/or ligation is within the scope of the invention. The term “hybrid” refers to a double-stranded nucleic acid molecule formed by hydrogen bonding between complementary nucleotides.

The term “complementary, “complement,” or “complementary nucleic acid sequence” refers to the nucleic acid strand that is related to the base sequence in another nucleic acid strand by the Watson-Crick base-pairing rules. In general, two sequences are complementary when the sequence of one can hybridize to the sequence of the other in an anti-parallel sense wherein the 3′-end of each sequence hybridizes to the 5′-end of the other sequence and each A, T(U), G, and C of one sequence is then aligned with a T(U), A, C, and G, respectively, of the other sequence. RNA sequences can also include complementary G/U or U/G basepairs.

The term “tag” as used herein, generally refers to a chemical moiety, which is used to identify a nucleic acid sequence, and preferably but not necessarily to identify a unique nucleic acid sequence. For instance, “tags” with different molecular weights can be distinguishable by mass spectrometry, and may be used to reduce the mass ambiguity between two or more nucleic acid molecules with different nucleotide sequences, but with the identical molecular weights. The “tag” may be covalently linked to an X-mer precursor, e.g., through a cleavable linker. “Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

A “hybridizing segment” is a region of an oligonucleotide that hybridizes with a target nucleic acid.

As used herein, “not genomically contiguous” means that the genomic binding sites of a first hybridizing segment of an oligonucleotide containing those segments (e.g., a first probe of a compound probe) and a second hybridizing segment (e.g., a second probe of a compound probe) of the oligonucleotide containing those segments are not contiguous. Non-genomically contiguous sequences may be separated by at least 5 bases, at least 100 bases, at least 1 kb, at least 10 kb, at least 100 kb and in certain cases may be on different chromosomes in a genome, e.g., a mammalian, e.g., human genome, etc.

A “signal” is a numerical measurement or an estimated (e.g., calculated) measurement of a characteristic of a signal received from scanning an array. Thus, a signal is a numerical score that quantifies some aspect of a spot/spot signal. For example, a mean intensity value of a spot is a statistic, as is a standard deviation value for pixel intensity within a spot. A signal can also refer to the “enrichment” of the probe, including, but not limited to, so-called “one-color” measurements, ratios between channels of a “two-color” assay, difference between channels of a “two-color” assay, or variants of these measures that are adjusted by normalization or by using estimates of the error in the measurements.

As used herein, “enrichment” refers to a signal or a meaningful combination of signals (e.g., of two colors of the same spot). For instance, in some embodiments, the scanner can measure two signal strengths for each feature: (1) the strength of a signal at a first wavelength that indicates the strength of the binding between the probes of a given feature and a control target; and (2) the strength of a signal at a second wavelength that indicates the strength of the binding between the probes of the aforementioned given feature and a test target. The ratio between the two signal strengths indicates the extent by which the test target differs from the control, and may indicate that a particular region of the genome is of interest. Thus, a high ratio between signal strengths from a test target and a control target (test:control) typically indicates a region of interest. The ratio is one of a number of possible ways of measuring the “enrichment” of the test target. Others include so-called “one-color” measurements (test), difference (test-control), or variants of these measures that are adjusted by normalization or by using estimates of the error in the measurements (test-control)/error. In certain embodiments, “signal” and “enrichment” are used interchangeably herein.

A “design file” may be provided by an array manufacturer and is a file that embodies some, or all, of the information that the array designer from the array manufacturer considered to be pertinent to array interpretation. Such a file may be written in XML and may describe the geometry as well as the biological content of a particular array.

As used herein, a “parameter” represents a definable characteristic of an item, device, or system. A parameter may refer to any data or information that describes the array, including but not limited to: the layout of the array, the spots, an array set, or probes within a compound probe. For example, the DNA sequence printed on a particular spot on the array is a parameter.

As used herein, “decoding” means to determine the meaning of a plurality of individual signals or data points. For example, in one embodiment, “decoding” includes identifying a particular nucleic acid sequence or a biological phenomenon using at least two or three addresses of an array.

### DETAILED DESCRIPTION

The present invention relates to systems and methods for processing to decode information from a microarray, and more specifically, to processing to decode information from a microarray comprising spots having multiple probes per spot. In one embodiment, spots of an array may include long probes (e.g., probes comprising greater than about 60 bases) in the form of compound probes. The compound probes can comprise at least first and second probes, including first and second nucleotide sequences capable of hybridizing to first and second target nucleotide sequences, respectively, in a nucleic acid molecule of interest. In other embodiments, some or all spots of an array can include multiple probes that are individually immobilized at the spot, rather than defining portions of compound probes. As such, a single spot of an array may include several different probes, which can increase the probe density of an array. The design of arrays and compound probes, in accordance with the invention—including two or more different probes (e.g., probes having different nucleic acid sequences)—can reduce the number of spots or arrays necessary to query the interactions of a large nucleic acid molecule of interest.

Although the description herein focuses primarily on deconvolution (or signal processing, as used interchangeably herein) of spot signals from spots comprising compound probes, it should be understood that the same techniques are applicable to deconvolution of spot signals from any spot comprising more than one probe. In such embodiments, each probe may be capable of contributing a signal to the spot signal, and the signals from the probes may be indistinguishable from one another. As such, a single spot signal may be an aggregated signal from all of the signals contributed by the probes of the spot. The probes of the spots may be attached to each other (e.g., covalently) in some embodiments, or non-attached to each other in other embodiments.

For example, a method comprising: a) reading an array comprising a plurality of spots that each contain nucleic acid sequences that hybridizes to non-contiguous genomic regions to identify spots that produce a signal; b) reading a design file to identify information on the sequences and/or the chromosomal binding sites of nucleic acid sequences; and c) decoding the information, is provided. In one embodiment, the array may contain oligonucleotide probes that comprise a plurality of hybridizing segments, wherein the hybridizing segments hybridize to non-contiguous regions in a target genome. In one embodiment, a hybridizing segment may be present at several positions on an array and may be part of different oligonucleotide at each position. As such, in certain cases the sequence that is common to the signal-producing spots of an array may be identified by looking up the addresses of signal-producing spots in a look-up table that provides the sequences of the oligonucleotides at those positions. In another embodiment, the hybridizing segments may be present at a single or multiple positions on an array and the sequence of a hybridizing nucleic acid (or the location of that nucleic acid in a genome) may be determined by looking up the addresses of signal-producing spots in a look-up table. In this embodiment, the look-up table may be used to identify which signal-producing sequences are adjacent but not contiguous to one another in a genome. The distribution of signal across those spots may identify the hybridizing sequence.

In one aspect, the invention provides techniques and equipment (e.g., software and related computer-associated components) for the analysis of chemical, biochemical, or biological assays. These techniques, articles, and systems are generally described in the summary of the invention above and, with an understanding of the assays from the description that follows, those of ordinary skill in the art can select suitable equipment and associated techniques to carry out the invention. In another aspect, a more specific set of techniques and related equipment is provided, and this is described in greater detail below, following a general description of the assays with which the present invention can be used.

Each of the following commonly-owned applications directed to related subject matter and/or disclosing methods and/or devices and/or materials useful or potentially useful for the practice of the present invention is incorporated herein by reference: a U.S. patent application filed on even date herewith, entitled “Compound Probes and Methods of Increasing the Effective Probe Density of Arrays,” by Leproust, et al.; a U.S. patent application filed on even date herewith, entitled “Methods of Increasing the Effective Probe Density of Microarrays,” by Gordon, et al.; and a U.S. patent application filed on even date herewith, entitled “Target Determination using Compound Probes,” by Sampas.

Multiple Probes, Including Compound Probes on Array Spots

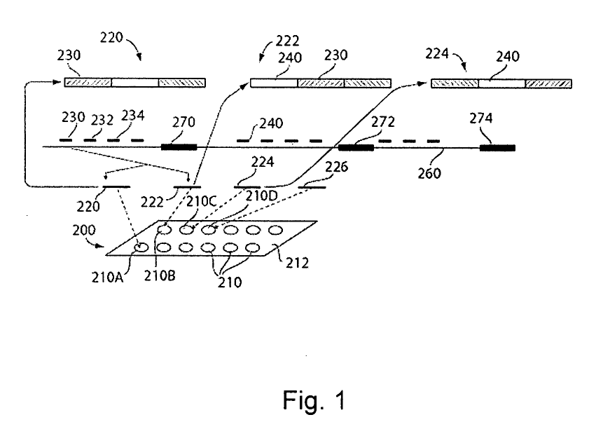

In embodiments of the invention, configurations and arrangements of probes within an array can vary. FIG. 1 shows one design of an array including spots comprising a homogenous composition of at least first and second probes. Spots 210 of array 200 may include multiple probes in the form of compound probes. For instance, compound probes 220, 222, 224, and 226 may be attached to surface 212 as individual spots 210A, 210B, 210C, and 210D, respectively. In this particular array, the set of compound probes is designed such that every probe of a compound probe is represented multiple times on array 200. For instance, each probe of a compound probe may be present on the array or array set as part of two different compound probes, and on two different spots of the array or array set. In other arrays, compound probes may be represented exactly two times, or exactly three times on an array or array set. Of course, the number of times a probe is represented on an array or array set may vary, e.g., depending on the design of the assay and/or the resolution of the assay. The degree of replication may vary within an array or array set; for example, an array (or array set) may have some probes that are present in two compound probes, some present in three, and some present in four or more.

In the embodiment illustrated in FIG. 1, the compound probes comprise three probes and each of the probes are represented at least twice as part of different compound probes of the array. The compound probes may be constructed randomly except for the constraint of representing each probe at least twice. In some cases, genomically nearby probes are not included on the same compound probe. For instance, compound probe 220 may include probe 230, which is located on, or near, gene 270 in nucleic acid molecule of interest 260. In one embodiment, the remaining two probes of compound probe 220 are not chosen from the group of probes on, or near, gene 270 (e.g., probes 232 and 234 are not a part of compound probe 220). Probe 230 of compound probe 220 may be represented on a different compound probe of the array; for instance, probe 230 may be present on compound probe 222, which is located on spot 210B of the array. Similarly, compound probe 222 may include 240, which is near gene 272. The remaining probe of compound probe 222 may be chosen from probes close to other genes, such as gene 274. Probe 240 may also be represented twice in the array, e.g., on two different compound probes of the array, such as on compound probe 224 in addition to compound probe 222. In some cases, an array or array set comprises at least two spots comprising a first oligonucleotide probe and at least two spots comprising a second of oligonucleotide probe, wherein the array or array set includes a first spot comprising the first and second oligonucleotide probes (e.g., as a compound probe) and does not include a second spot comprising the first and second oligonucleotide probes. In other cases, the array or array set includes a first spot comprising the first and second oligonucleotide probes and a second spot comprising the first, but not the second oligonucleotide probe. The array or array set can further comprise a third spot that comprises the second oligonucleotide probe but not the first oligonucleotide probe. For example, if a first and a second oligonucleotide probe are included in one spot in the array or array set, then those first and second probes would not normally co-occur on any other spot in the array or array set.

Array 200 of FIG. 1 may be contacted with a sample under conditions that permit hybridization between target nucleotide sequences of the sample and sequences of the oligonucleotide probes. After hybridization and scanning, one or more spots may fluoresce to produce spot signals. In some cases, to determine which probe contributed to the spot signal (e.g., to determine which of the probes of the compound probe the target nucleotide sequence was hybridized), the signal from one spot may be correlated to the signal from another spot. For instance, if spot 210A produced a signal (e.g., a spot signal), it may be useful to look at signals from spots 210B and 210C to determine which probes contributed to the spots signals.

First Exemplary Technique for Deconvolution; Analysis of Data Associated with Assays

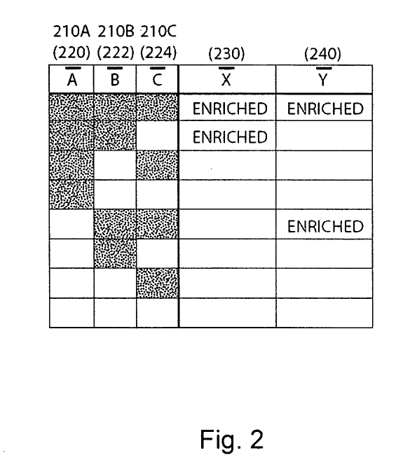

As shown in FIG. 2, each of spots 210A, 210B, and 210C can each produce signals (illustrated by the shaded areas in FIG. 2). Since it is known where each probe of a compound probe is located on the array or array set, to determine whether probe 230 contributed to the probe signal of spot 210A (compound probe 220), one can observe whether a similar signal was obtained from spot 210B, which also includes probe 230. In cases when 210A and 210B both produced signals (as in the first two rows of the table), it is likely that 230 contributed to the signals of these spots because both of these spots include probe 230. Similarly, to determine whether probe 240 of spot 210B (compound probe 222) contributed to the spot signal, one can observe whether a similar signal was obtained from spot 210C, which also includes probe 240. If spots 210B and 210C produced signals (as in the first and fifth rows of the table), and both of these spots include probe 240, it is likely that 240 contributed to the signals of these spots. In some cases, a signal of a probe is considered significant if all of the compound probes including that probe sequence show a significant signal. In one particular embodiment, the biological phenomenon is identified if and only if all of the spots comprising probes relating to that phenomenon show a signal. In another embodiment, a significance can be computed for a biological phenomenon at a particular probe based on the significance of the signals of each of the compound probes including that probe, by, for example, computing the joint-likelihood of the pair of signals. Accordingly, enrichment or hybridization of a probe with a target, and the contribution of a signal from one probe among a plurality of probes within a compound probe, can be determined. As such, multiple signals from multiple spots can be correlated to determine the location of a biological phenomenon in terms of chromosomal coordinates in the nucleic acid molecule of interest.

Second Exemplary Technique for Deconvolution; Analysis of Data Associated with Assays

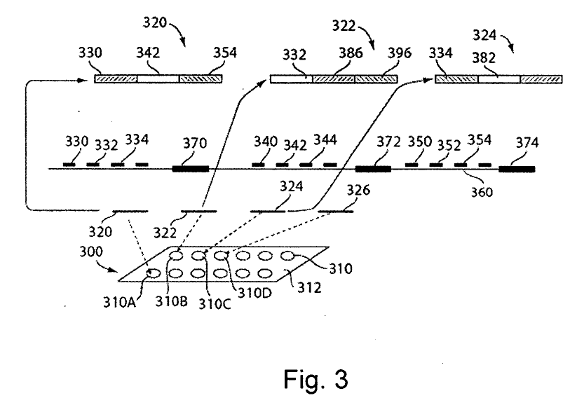

The embodiment illustrated in FIG. 3 shows another arrangement of compound probes on an array, wherein each compound probe comprising three probes. Of course, in other embodiments, each compound probe can comprise any suitable numbers of probes (e.g., four, five, six, or more probes). In one embodiment, each of the probes is represented once in the array (or array set) and probes that are genomic neighbors (or are nearby) are not included on the same compound probe. The compound probes may be constructed randomly, except for these constraints. For instance, compound probe 320 may include probe 330, which is located on, or near, gene 370 in nucleic acid molecule of interest 360. In one embodiment, the remaining two probes of compound probe 320 are chosen randomly, except they are not chosen from the group of probes on, or near, gene 370 (e.g., probes 332 and 334 are not a part of compound probe 320). Instead, the remaining two probes of compound probe 320 may be chosen from the group of probes on, or near, other genes such as gene 372 and/or 374. For example, compound probe 320 may include probe 342, which is on or near gene 372, and probe 354, which is on or near gene 374.

Since in this particular assay, each probe is presented only once, compound probe 322 can have a unique combination of probes compared to compound probe 320. For example, each of the probes of compound probe 322 may be chosen randomly from different portions of nucleic acid molecule of interest 360, each portion being on or near different genes relative to the other portions. Advantageously, such an array can increase the effective resolution of an array by a factor equal to the number of probes in each compound probe. For example, for compound probes having three probes (e.g., as shown in FIG. 3), the effective resolution can increase by a factor of three. Similarly, for compound probes having n probes, the effective resolution can increase by a factor of n.

Arrays (e.g. the array 300 of FIG. 3) may be contacted with a sample under conditions that permit hybridization between target nucleotide sequences of the sample and sequences of the oligonucleotide probes. After hybridization and scanning, one or more spots may fluoresce to produce spot signals. In some cases, it may be desirable to determine which probe contributed to the spot signal (e.g., to determine which of the probes of the compound probe the target nucleotide sequence was hybridized). In other cases, however, it is not necessary to determine which probe contributed to the spot signal in order to determine the location of a biological phenomenon in terms of chromosomal coordinates in the nucleic acid molecule of interest. In some embodiments, the signal from one spot may be correlated to the signal from one or more other spots in order to determine the location of the biological phenomenon.

In one embodiment, the constraints of having probes that are non-genomic neighbors of one another on the same compound probe can aid in the deconvolution (e.g., processing) of signals obtained upon hybridization. In some cases, knowledge of the expected correlation between neighboring probes can also help in deconvoluting the contribution of each probe of a compound probe from a spot signal.

In some cases, the signal associated with a biological phenomenon at a specific location on a nucleic acid molecule of interest is distributed to probes that are genomic neighbors. For instance, since fragmentation of the nucleic acid of interest is performed randomly, fragments including different nucleotide sequences may include the same signal associated with the biological phenomenon. When the fragment length exceeds the probe spacing (in genomic coordinates), a biological phenomenon can generate a signal that is spread across a set of probes in a genomic region. For example, if the median fragment length is about 800 bp and the average probe spacing is about 30 bp, then a given biological phenomenon can contribute a signal across a genomic “neighborhood” of about 26 probes (e.g., 800 bp divided by 30 bp spacing). Some of the embodiments presented here use this expected correlation among probes that are genomic neighbors for the deconvolution of signals from compound probes.

In one embodiment, processing or deconvolution of signals obtained upon hybridization may be performed at least in part by the fragment distribution, which can be generally approximated (e.g., about 800 bp fragments for a typical ChiP-chip sonication protocol) or inferred (e.g., from precise measurement of individual samples via gel electrophoresis or an Agilent Bio-Analyzer). Deconvolution can be achieved by analyzing a spot signal of compound probes in the genomic context of the probes making up the compound probes. For example, if a particular compound probe including a first and a second probe produces a spot signal, then it can be determined which probe of the compound probe is/are responsible for the signal by looking at the spot signal in the context of the signals of the other compound probes comprising the genomic neighbors of the first probe, and then repeating for the second probe, and so on. The analysis of an expected distribution can take on many forms, e.g., ranging from peak-fitting (e.g., of intensities and/or ratios) to a more comprehensive error model that takes into account the error in the probe intensities and/or knowledge of the expected signal distribution. Such an error model can propagate these errors to make a final estimate of the confidence in identifying signal-producing regions.

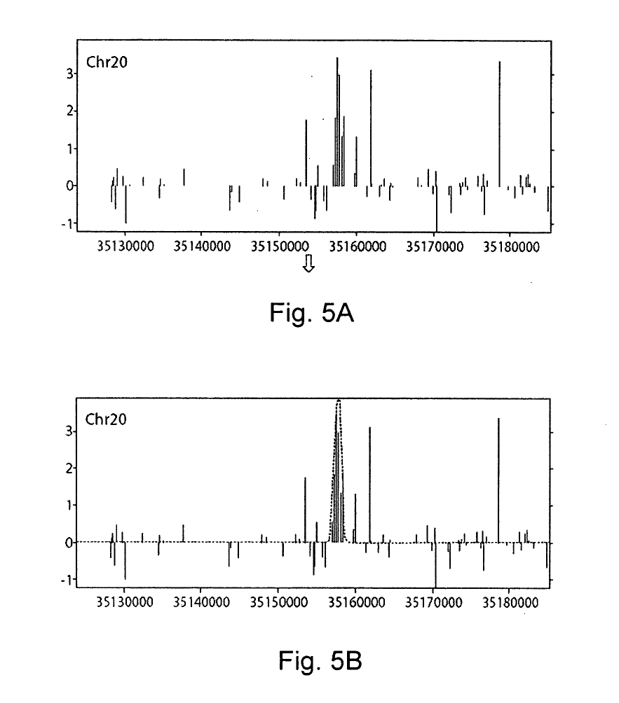

An example of discerning which probe of a compound probe is responsible for a spot signal can be shown in reference to FIG. 3. Since it is known where each probe of a compound probe is located on the array or array set, to determine whether probe 330 contributed to the signal of spot 310A (compound probe 320), one can observe whether signals were obtained from the genomic neighbors of probe 330. For example, signals from spots comprising probes 332 and 334 (e.g., spots 310B and 310C, respectively) may be analyzed together with the signal from spot 310A, because the signal associated with a biological phenomenon at a particular location on nucleic acid of interest 360 may be distributed to probes that are genomic neighbors. In some cases, if the signals arising from probes that are genomic neighbors form an expected distribution of signals (e.g., a Gaussian distribution), the presence of the expected distribution may indicate the location of the biological phenomenon, e.g., at the peak of the distribution. The fitting of the shape of the distribution to signals are shown, for example, in FIGS. 5A and 5B. Note that in this example, no fit is found for probes exhibiting high signals inconsistent with neighboring probes. The absence of an expected distribution may indicate the absence of a biological phenomenon at that particular location. Similarly, probe 342 of compound probe 320 may be analyzed in connection with the genomic neighbors of probe 342 (e.g., probes 340 and 344), and the distribution of signals across those genomic neighbors may indicate the presence or absence of a biological phenomenon at that particular location along nucleic acid molecule of interest 360. Accordingly, in one embodiment, the biological phenomenon is identified if and only if all of the spots comprising probes in the genomic neighborhood of the phenomenon show a signal.

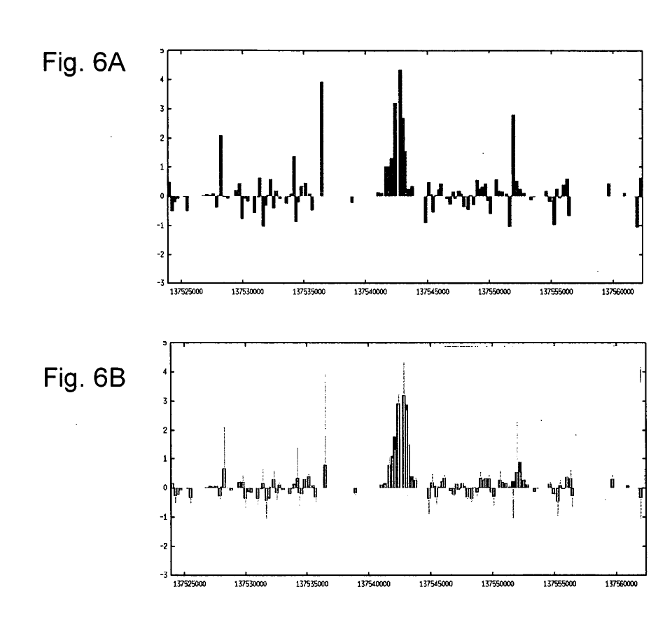

In addition to the method described above, if desired, post-processing can be performed on signals before fitting to a shape of a distribution. For instance, signals can be passed through a filter constructed to reduce high signals that are inconsistent with neighboring signals, as may occur when only one of the probes in a compound probe is associated with a bona-fide biological event. Such filtering is shown in FIGS. 6A and 6B, using an expression such as:

$${S}_{low - pass} = wt ( {S}_{i} ) {S}_{i} + \frac{1 - wt ( {S}_{i} )}{2} ( {S}_{i - 1} + {S}_{i + 1} )$$

$$wt ( S ) = \frac{1}{1 + abs ( S )}$$

where the signal S_{processed,i}, (or intensity or ratio) that is to be associated with a probe i, is computed as a weighted average of the unfiltered signal of the probe with the unfiltered signals of the probes preceding it Sᵢ₋ₗ, and following it Sᵢ₊ₗ. In this example, the weighting is inversely proportional to the absolute value of the log-ratio of the probe.

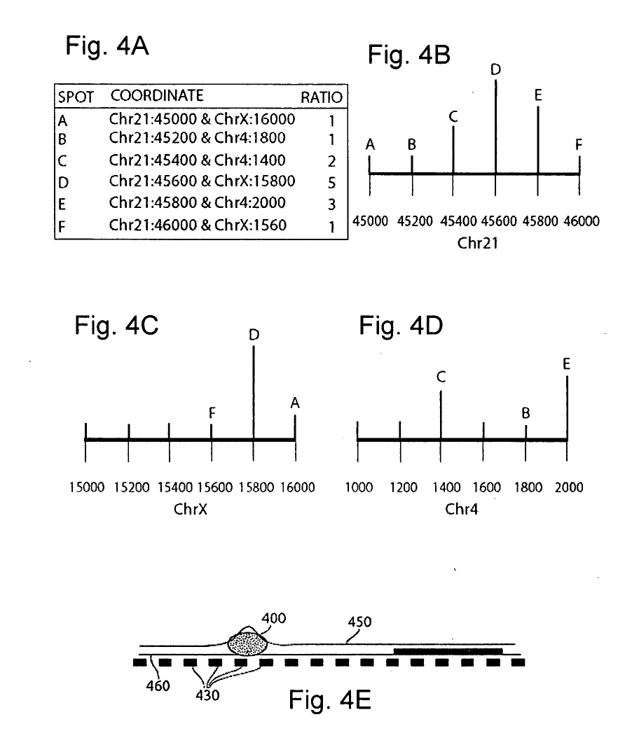

In another example, in an array including compound probes comprising first and second probes that are not genomic neighbors on the nucleic acid molecule of interest, each spot generates one spot signal, but this one signal can give useful information about two particular positions on the nucleic acid molecule of interest. (Similarly, a compound probe including three probes can produce one signal that can give useful information about three particular positions on the nucleic acid molecule of interest.) As illustrated in one example shown in FIG. 4A, spot A includes a first probe located on Chr21 at base number 45,000, and a second probe located on chromosome X (ChrX) at base number 16,000 on the nucleic acid molecule of interest. The signal of spot A, which may be shown as a ratio of signals (e.g., a ratio of the spot signal to a base signal), may be plotted along the coordinates of the nucleic acid molecule of interest, e.g., as shown in FIGS. 4B and 4C. Similarly, spot B comprising a first probe located on Chr21 at base number 45,200, and a second probe located on chromosome 4 (Chr4) at base number 1,800 can produce a signal with a ratio of 1 that can be plotted as shown in FIGS. 4B and 4D. A similar approach can be followed for all of the spots of the array, and each signal may be evaluated in connection with the signals from genomic neighbors. In such cases, a signal of a probe may be considered significant if the compound probes which include probes that are genomic neighbors show a significant or expected signal.

As shown in the embodiment illustrated in FIG. 4, spot D produces a signal with a ratio of 5, which may indicate that a biological phenomenon is associated with the probes that make up the compound probe of spot D. In order to determine which probe contributed to the signal at spot D, the signals of the genomic neighbors of the probes of spot D may be analyzed, e.g., as shown in FIGS. 4B and 4C. FIG. 4B shows an expected distribution of signals around Chr21 at base number 45,600, which indicates that the biological phenomenon is likely associated with that position on the nucleic acid molecule of interest. In contrast, the distribution of signals shown in FIG. 4C, is not consistent with an expected distribution, which implies that the biological phenomenon is likely not associated with ChrX at base number 15,800. Advantageously, the signals arising from neighboring probes can be used to differentiate signal (e.g., hybridization on Chr21 at base number 45,600) from noise (e.g., hybridization on ChrX at base number 15,800). FIG. 4E shows the relationship between a biological phenomenon, indicated here by the binding of transcription factor 400 with nucleic acid molecule of interest 460, and probes 430 that give rise to signal 450.

In another embodiment, additional deconvolution or decoding or signals can be achieved by substituting surrogate base-line measurements for probes at certain locations in cases where high signals are attributed to phenomenon at other genomic locations. As described above in connection with FIG. 4, for example, the high enrichment of probe “D” representing locations on Chr21 (FIG. 4B) and ChrX (FIG. 4C) is attributed to the location on Chr21 because its genomic neighbors at that location exhibit the expected distribution. As this attribution is made, the high enrichment at the location on ChrX can be replaced with a base-line value, such as a ratio of one, in order to facilitate further analysis. After the substitution is made, the enrichment at the associated position on ChrX in FIG. 4C will be low (log-ratio of 0), while the enrichment at the associated position on Chr21 in FIG. 4B will be preserved.

It should be understood that while the description herein involves using separate processing or deconvolution methods for each array or array set, in other embodiments, two or more such techniques can be used in conjunction for a single array or array set. For example, in one embodiment, an array or array set can involve both the use of replicate probes and genomic adjacency. In such instances, the deconvolution methods can depend on both replication (for particular probes that were replicated) and genomic adjacency to determine underlying biological events.

Computer-Related Techniques

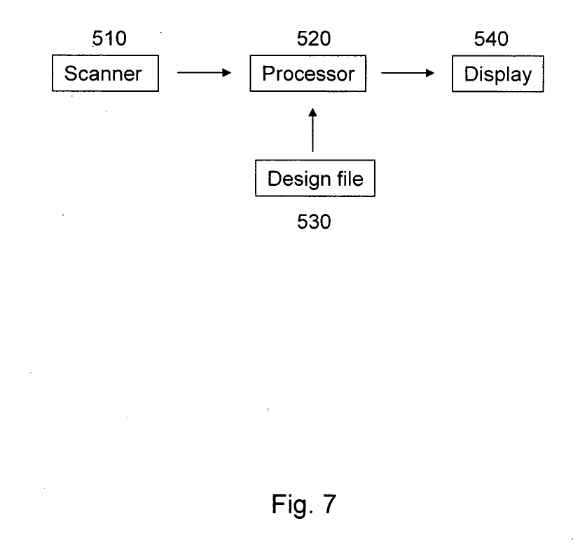

Referring now to FIGS. 5 and 6, one set of embodiments for specific implementation of assay interpretation will be described. FIG. 7 is a schematic diagram showing the interrelationship between an array scanner 510, a processor 520, a design file 530, and a display 540. In use, scanner 510 obtains information on the condition of one or more spots of an array. The scanner can determine the condition of the array based upon one or more of a variety of characteristics, for example, color of various spots, fluorescent signal of various spots, other radiative characteristic of various spots, mass of various spots, or any other phenomena indicating that a spot has experienced a chemical, biological, or biochemical event to be determined, for example, a specific biological event. The scanner, in one embodiment, produces a set of digital signals representative of the condition of the array including some or all spots, and communicates this information to processor 520. Design file 530 contains actuatable information (e.g., instructions input by an operator, instructions loaded in association with a file, instructions introduced via pre-prepared software, or the like) and communicates with processor 520 so that the processor can manipulate data, from scanner 510, and provide an analysis in a form suitable for display 540. The analysis, when transferred to display 540, can be displayed in a form useful by the user. For example, the display can be a picture showing spots of an array and indicating what biological events took place at those spots (e.g., which chemical species bound to those spots), a picture showing which species bound to which specific probes were located at those spots, where “picture” can be a physical picture or other pictoral representation, or the like. Alternatively, data can be displayed in written form, tabular form, or any other form with this information. In another arrangement, the display simply communicates to the user which species bound to which probes, regardless of where those probes occurred on the array or which species bound to which spots on the array. The arrangement of FIG. 7 can be used to analyze information according to any of the techniques described above.

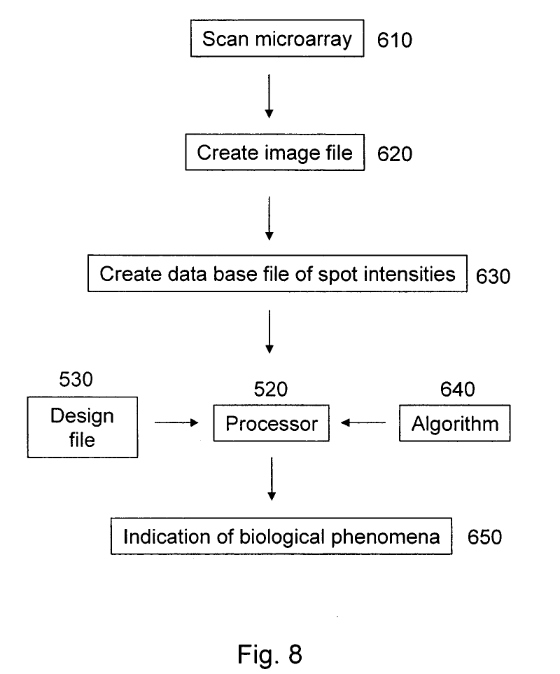

Referring now to FIG. 8, additional details of the analysis process are described, including acts associated with the process. In FIG. 8, an act 610 involves scanning a microarray, i.e., reading spot signals associated with spots of an array or array set, for example, using a scanner 510. Then, an image file can be created (620) which can be a digitized set of information produced by scanner 510 in association with the step 610 of scanning the microarray. The image file typically is digitized information associated with a visual scanning process, e.g., digital representation of color of array spots, fluorescence of array spots, other radiative or other indicative signals produced by array spots, etc. The image file may represent one, two or more channels (e.g., two-color or multi-colored channels) representing the signal obtained different scanning wavelengths. Those of ordinary skill in the art are aware of the types of signals that can be produced by such array spots and how such an image file can be created.

Then, in the embodiment illustrated in FIG. 8, a database file of spot intensities can be created according to act 630. This step can be carried out in association with and essentially simultaneously with (e.g., in the same act as) act 620, or can be carried out separately. The database file of spot intensities can be stored electronically in a computer, can be written to a storage medium such as a disc, magnetic storage medium, or the like, which may or may not be removable from and reintroducable to a computer associated with creation of the image file or creation of the database file, or another computer. Those of ordinary skill in the art are well aware of suitable storage media or other equipment useful for creating a database file of spot intensities.

In some embodiments, a machine-readable medium having a program stored thereon is provided. The program can instructions for, when executed, performing acts of analyzing values of spot signals associated with spots of an array or array set, wherein each spot signal, for at least some of the spot signals, is associated with at least first and second oligonucleotide probes. In some embodiments, a value of a first spot signal corresponds to the first oligonucleotide probe only if the value of the first spot is statistically significant, and if a value of at least a second spot signal from at least a second spot, which comprises the first, but not the second, oligonucleotide probe, is also statistically significant. In another embodiment, a value of a first spot signal corresponds to the first oligonucleotide probe only if the joint significance of the value of the first spot and a value of at least a second spot signal from at least a second spot, which comprises the first, but not the second, oligonucleotide probe, is significant. In other embodiments, a value of a first spot signal corresponds to the first probe only if values of spot signals from spots comprising probes that are genomic neighbors of the first probe, together with the value of the first spot signal, produce an expected distribution of values.

The database file can then be shared with processor 520 which can interact with design file 530 as described above and can be driven by an algorithm 640 to be implemented in association with processor 520 to produce an indication of biological phenomenon 650, which can be embodied in association with a display 540, or in another arrangement. For example, indication 650 can involve a physical print-out, data stored on a storage medium as described above, visual display on a computer screen, or the like. The algorithm 640 can be written, by those of ordinary skill in the art, using such examples as those shown in Kim et al, “A high-resolution map of active promoters in the human genome,” Nature, 436, 876-880, (2005); M. Zheng, L. Barrera, B. Ren, and Y. Wu, “ChIP-chip: Data, Model, and Analysis,” Paper 2005102801, Department of Statistics, eScholarship Repository, UCLA, (2005); and T. Kaplan & N. Friedman, “Model-Based Analysis of High-Resolution Chromatin-Immunoprecipitation,” Technical Report 2006-11, School of Computer Science & Engineering, Hebrew University, 2006, with the benefit of the disclosure herein concerning various assays and analysis thereof. For example, algorithm 640 can be written so as to carry out deconvolution as set forth in connection with FIGS. 1-4, and related description above.

In another arrangement, data can be scanned, and analyzed, in different settings, by different parties, and/or using different equipment. For example, an assay product can be scanned as described above and this information stored in a storage medium, transmitted over the internet, or otherwise held in a useful form. At a different location, a different point in time, and/or with a different party involved, the data can then be analyzed. Analysis can be in a separate module of hardware and software relative to that of the scanning equipment. Those of ordinary skill in the art are readily aware of how the scanning and analysis features of the invention as described above can be separated in time, space, and/or personnel.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method comprising:
a) reading an array comprising a plurality of spots that each contain nucleic acid sequences that hybridize to non-contiguous genomic regions to identify spots that produce a signal;
b) reading a design file to identify information on the sequences and/or the chromosomal binding sites of nucleic acid sequences; and
c) decoding said information to identify a biological phenomenon.

2. A method as in claim 1, wherein said plurality of spots comprise a mixture of oligonucleotides having different sequences.

3. A method as in claim 1, wherein said plurality of spots comprise an oligonucleotide comprising a plurality of hybridizing segments.

4. A method as in claim 3, wherein the oligonucleotide includes at least a first and a second hybridizing segment that are contiguous on the oligonucleotide.

5. A method as in claim 3, wherein the oligonucleotide includes at least a first and a second hybridizing segment that are not contiguous on the oligonucleotide.

6. A method as in claim 3, wherein the oligonucleotide is at least 100 nucleotides in length.

7. A method as in claim 3, wherein the hybridizing segments are each at least 40 bases in length.

8. A method as in claim 3, wherein the oligonucleotide comprises at least 3 hybridizing segments.

9. A method, comprising acts of:
reading spot signals associated with spots of an array or array set, wherein each spot, for at least some of the spots of the array or array set, include at least first and second oligonucleotide probes having respective nucleotide sequences;
reading a design file comprising parameters including the nucleotide sequence of each probe within said spots of the array or array set, and/or the location of each probe in terms of chromosomal coordinates if the probes were hybridized to a nucleic acid molecule of interest;
associating a corresponding spot signal with a parameter from the design file; and
for at least some of the spots including those corresponding to first and second oligonucleotide probes per spot, processing to decode information identifying a biological phenomenon in the nucleic acid molecule of interest.

10. A method as in claim 9, wherein the biological phenomenon can be the presence or absence of protein binding on the nucleic acid molecule of interest.

11. A method as in claim 9, further comprising, from one or more readings of spot signals, creating a file including values of spot signals associated with the spots.

12. A method as in claim 9, wherein at least a plurality of spots of the array include compound oligonucleotide probes.

13. A method as in claim 12, wherein the compound probes have an average length of at least 100 nucleotides.

14. A method as in claim 13, wherein at least a plurality of the compound probes comprise at least first and second oligonucleotide probes contiguous on the compound probe.

15. A method as in claim 9, wherein a plurality of the spots comprise multiple, non-contiguous probes.

16. A method as in claim 9, wherein the biological phenomenon is identified if and only if all of the spots comprising probes relating to that phenomenon show a signal.

17. A method as in claim 9, wherein the biological phenomenon is identified if and only if all of the spots comprising probes in the genomic neighborhood of the phenomenon show a signal.

18. An article, comprising:
a machine-readable medium having a program stored thereon, which program has instructions for, when executed, performing acts of:
analyzing values of spot signals associated with spots of an array or array set, wherein each spot signal, for at least some of the spot signals, is associated with at least first and second oligonucleotide probes, and wherein a value of a first spot signal corresponds to the first oligonucleotide probe only if the joint significance of the value of the first spot and a value of at least a second spot signal from at least a second spot, which comprises the first, but not the second, oligonucleotide probe, is significant.

19. An article, comprising:
a machine-readable medium having a program stored thereon, which program has instructions for, when executed, performing acts of:
analyzing values of spot signals associated with spots of an array or array set, wherein each spot signal, for at least some of the spot signals, is associated with at least first and second oligonucleotide probes, and wherein a value of a first spot signal corresponds to the first probe only if values of spot signals from spots comprising probes that are genomic neighbors of the first probe, together with the value of the first spot signal, produce an expected distribution of values.

20. A system, comprising:
a scanner for reading spot signals from an array or array set including a plurality of spots, wherein each spot, for at least some of the spots of the array or array set, include at least first and second oligonucleotide probes having respective nucleotide sequences; and
a processor for receiving output from the scanner and executing operations to analyze the scanner output and providing an indication of a biological phenomenon in a nucleic acid molecule of interest.
